(19) United States
(12) Patent Application Publication
(10) Pub. No.: US 2009/0004119 A1
(43) Pub. Date: Jan. 1, 2009

(54) Polymers

(75) Inventors: Alan Cuthbertson, Oslo (NO); Andreas Meijer, Oslo (NO); Bente E. Arbo, Bekkestua (NO); Magne Solbakken, Skien (NO)

Correspondence Address: GE HEALTHCARE, INC., IP DEPARTMENT, 101 CARNEGIE CENTER, PRINCETON, NJ, 08540-6231, US

(73) Assignee: GE HEALTHCARE AS, Oslo (NO)

(21) Appl. No.: 12/146,529

(22) Filed: Jun. 26, 2008

(30) Foreign Application Priority Data
Jun. 27, 2007 (NO) 20073313

(51) Int. Cl. A61K 49/06; C08G 63/00; A61K 49/00

(52) U.S. Cl. 424/9.361; 528/271; 424/9.1

(57) ABSTRACT

The present invention relates to novel linear polymers, compositions comprising said polymers and their use as contrast agents in magnetic resonance (MR) imaging (MRI) and magnetic resonance spectroscopy (MRS).

### FIELD OF THE INVENTION

The present invention relates to novel linear polymers, compositions comprising said polymers, processes for preparing said polymers and their use as contrast agents in magnetic resonance (MR) imaging (MRI) and magnetic resonance spectroscopy (MRS).

### BACKGROUND OF THE INVENTION

MR image signal is influenced by a number of parameters that can be divided into two general categories: inherent tissue parameters and user-selectable imaging parameters.

Inherent tissue parameters that affect MR signal intensity of a particular tissue are mainly the proton density, i.e. hydrogen nuclei density of that tissue and its inherent T₁ and T₂ relaxation times. Signal intensity is also influenced by other factors such as flow. The contrast between two adjacent tissues, e.g. a tumour and normal tissue depends on the difference in signal between the two tissues. This difference can be maximised by proper use of user-selectable parameters. User-selectable parameters that can affect MR image contrast include choice of pulse sequences, flip angles, echo time, repetition time and use of contrast agents.

Contrast agents are often used in MRI in order to improve the image contrast. Contrast agents work by effecting the T₁, T₂ and/or T₂* relaxation times and thereby influencing the contrast in the images. Information related to perfusion, permeability and cellular density as well as other physiological parameters can be obtained by observing the dynamic behaviour of a contrast agent.

Several types of contrast agents have been used in MRI. Water-soluble paramagnetic metal chelates, for instance gadolinium chelates like Omniscan™ (GE Healthcare) are widely used MR contrast agents. Because of their low molecular weight they rapidly distribute into the extracellular space (i.e. the blood and the interstitium) when administered into the vasculature. They are also cleared relatively rapidly from the body.

Blood pool MR contrast agents on the other hand, for instance superparamagnetic iron oxide particles, are retained within the vasculature for a prolonged time. They have proven to be extremely useful to enhance contrast in the liver but also to detect capillary permeability abnormalities, e.g. “leaky” capillary walls in tumours which are a result of tumour angiogenesis.

The existent paramagnetic metal chelates that are used as MR contrast agents have a low relaxivity at the 1.5 T magnetic fields that is standard in most of today's MR scanners. In 3 T systems which probably will dominate or at least be a substantial fraction of the market in the future, the intrinsic contrast is lower, all T₁ values are higher and the hardware will be faster, so the need for a contrast agent with good performance at 3 T is considerable. In general, the longitudinal relaxivity (r1) of contrast agents falls off at the high magnetic fields of the modern MR scanners, i.e. 1.5 T, 3 T or even higher. This is due to the fast rotational Brownian motion of small molecules in solution which leads to weaker magnetic field coupling of the paramagnetic metal ion to the water molecules than anticipated.

Many attempts have been made to produce contrast agents with high relaxivity by incorporating the paramagnetic metal chelates into larger molecules, such as various polymers. All these attempts have been of limited success because of fast internal rotations or segmental motions.

W0-A2-2003/014157 discloses conjugates of peptides and metal complexes which are used as MRI contrast agents.

US 2006/0140869 (General Electric Company) discloses a Gd-based polypeptide with improved relaxivity as a result of minimized internal flexibility due to the introduction of steric hindrance molecules incorporated onto the polymeric backbone. The polymeric backbone described in this patent application is based on flexible amino acids such as lysine and ornithine.

US 2006/0104908 (General Electric Company) discloses a synthetic method based on polymerization of Gd-chelate conjugated N-carboxyanhydride monomers. The polymeric structures described in this patent application are based on flexible amino acids such as lysine and ornithine.

Aime et al., Chem Commun, 1999, 1577-1578 demonstrates the importance of secondary structure in Gd-based polylysine polymers by showing that increasing pH induces a change in the structure of the macromolecules resulting in increased relaxivity.

### SUMMARY OF THE INVENTION

It is a need of solving the abovementioned problems by providing novel compounds that perform well as MR contrast agents at high magnetic fields, i.e. magnetic fields above 1.5 T.

### DETAILED DESCRIPTION OF THE INVENTION

The present invention solves said problems by providing novel compounds consisting of linear polymers comprising conformationally constrained amino acids where the free rotation in the side chain is restricted. This additional level of constraint provides relaxivity equivalent to large macromolecules but with considerably fewer amino acids per molecule.

Thus in a first aspect the present invention provides a linear polymer comprising from 4 to 100 units of the formula (I)

A-L-X (I)

wherein
A is the same or different and is a conformationally constrained amino acid residue;
L is absent or present and is the same or different and denotes a linker moiety;
X is the same or different and denotes a chelator; and
wherein said units are linked to each other by amide bonds between said As.

In linear polymers comprising units of formula (I), A is preferably the same conformationally constrained amino acid residue, L absent or present and if present, L is preferably the same linker moiety and X is preferably the same chelator.

The term “chelator” denotes a chemical entity that binds (complexes) a metal ion to form a chelate. If the metal ion is a paramagnetic metal ion, the chemical entity, i.e. complex, formed by said paramagnetic metal ion and said chelator is denoted a “paramagnetic chelate”.

In a preferred embodiment the present invention provides linear polymers comprising from 4 to 100 units of the formula (II)

A-L-X′ (II)

wherein
A is the same or different and is a conformationally constrained amino acid residue;
L is absent or present and is the same or different and denotes a linker moiety;
X′ is the same or different and denotes a paramagnetic chelate consisting of a chelator X and a paramagnetic metal ion M; and
   wherein said units are linked to each other by amide bonds between said As.

In said embodiment of the present invention, said paramagnetic chelate consists of the chelator X and a paramagnetic metal ion M, said chelator X and paramagnetic metal ion M form a complex which is denoted a paramagnetic chelate.

In linear polymers comprising units of formula (II), A is preferably the same conformationally constrained amino acid residue, L absent or present and if present, L is preferably the same linker moiety and X′ is preferably the same paramagnetic chelate.

When the linear polymers of the present invention are used as MR high relaxivity agents the polymers preferably comprise from about 4 to 20 units of the formula (II), more preferably from about 4 to 10. MR imaging media for tumour imaging preferably comprise linear polymers comprising from about 5 to 70 units of formula (II), more preferably from about 10 to 30. When the linear polymers are used as MR blood pool agents the linear polymers preferably comprise from about 10 to 60 units of formula (II), more preferably from about 15 to 40.

In a preferred embodiment, A is an α,α-substituted amino acid.

In a more preferred embodiment A is of the general formula (III)

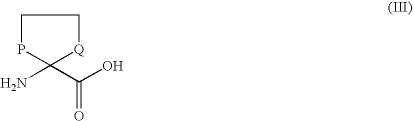

wherein
P is absent or is —CH₂— or —(CH₂)ₙ—NH—; where n is 0 to 6
Q is absent or is —CH₂— or —(CH₂)ₙ—NH—; where n is 0 to 6
where at least one of P or Q is present and is —(CH₂)ₙ—NH—

A preferred example of said formula (III) is

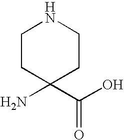

Another preferred example of formula (III) is

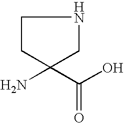

Another preferred embodiment A is of the general formula (IV)

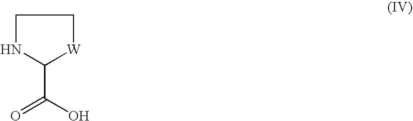

wherein
W is —(CH₂)ₙ—NH—; where n is 0 to 6

A preferred example of said formula (IV) is

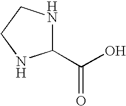

Another preferred example of formula (IV) is

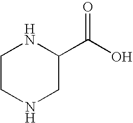

The linear polymers according to the present invention may further comprise spacers that can be introduced into the polymers between the amino acid residues A of the units (I) and (II) by linking said spacers to said As by amide bonds. Suitable spacers are any conformationally constrained amino acids, for example α,α dimethyl alanine. Said spacers can be introduced to provide a distance between said units ensuring enough space for said chelators or chelates. Said spacers can preferably be introduced between each of the units (I) or (II) in the polymer, but spacers can also be introduced with a certain number of units (I) or (II) between said spacers or at random frequency through the polymer.

In linear polymers according to the present invention, L may be present or not. If L is present, each L is the same or different and denotes a linker moiety, i.e. a moiety that is able to link A and X or A and X′, respectively. If L is not present, A is directly attached to X (units of formula (I)) or X′ (units of formula (II)) via an amide bond.

Preferred examples of L are:

Linker moieties —(CZ¹Z²)ₘ—
wherein
m is an integer of 1 to 6; and
Z¹ and Z² independently of each other denote a hydrogen atom, a hydroxyl group or a C₁-C₈-alkyl group optionally substituted by hydroxyl, amino or mercapto groups, e.g. CH₂OH and CH₂CH₂NH₂ and/or optionally comprising an oxo-group, e.g. CH₂OCH₃ and OCH₂CH₂OH.
   Linker moieties *—CO—N(Z³)
   wherein
* denotes the attachment of A to said linker moiety; and
Z³ stands for H, C₁-C₈-alkyl, optionally substituted with one or more hydroxyl or amino groups.
   Linker moieties *—CZ¹Z²-CO—N(Z³)- which are preferred linker moieties,
   wherein
* denotes the attachment of A to said linker moiety;
Z¹ and Z² have the meaning mentioned above; and
Z³ stands for H, C₁-C₈-alkyl, optionally substituted with one or more hydroxyl or amino groups.

In a preferred embodiment, Z¹ and Z² are hydrogen or Z¹ is hydrogen and Z² is methyl and Z³ is H or C₁-C₃-alkyl, e.g. methyl, ethyl, n-propyl or isopropyl, optionally substituted with one or more hydroxyl or amino groups, e.g. CH₂OH, C₂H₄OH, CH₂NH₂ or C₂H₄NH₂.

Linker moieties which are amino acid residues *—CZ¹Z²-CO—NH—CH(O)CO—NH—
wherein
* denotes the attachment of A to said linker moiety;
Z¹ and Z² have the meaning mentioned above, preferably Z¹ and Z² are hydrogen or Z¹ is hydrogen and Z² is methyl; and
Z⁴ stands for the side group of the naturally occurring α-amino acids.
   Linker moieties *—CO—NH—CZ¹Z²—
   wherein
* denotes the attachment of A to said linker moiety; and
Z¹ and Z² have the meaning mentioned above, preferably Z¹ and Z² are hydrogen or Z¹ is hydrogen and Z² is methyl

Further preferred examples of L comprise benzene or N-heterocycles such as imidazoles, triazoles, pyrazinones, pyrimidines and piperidines, where A is attached to a carbon atom in said N-heterocycles or in benzene.

If L comprises benzene, L is preferably

-*benzene-(CZ¹Z²)ₘ-
wherein
A is attached to a carbon atom in said benzene; and
Z¹, Z² and m are as defined above.

A preferred example of L, wherein * denotes the attachment of A to said linker moiety is:

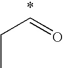

In preferred embodiments of the linear polymers according to the present invention, X is X′ which stands for a paramagnetic chelate, i.e. a chelator X which forms a complex with a paramagnetic metal ion M. X′ is the same or different. Preferably, all X′ are the same.

Numerous chelators X which form complexes with paramagnetic metal ions M are known in the art. Preferably, X is a cyclic chelator of formula (V):

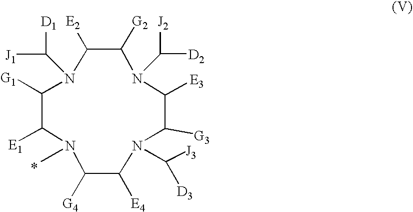

wherein
* denotes the attachment of L
E₁ to E₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃ or C(O)N(CH₂CH₃)₂;
G₁ to G₄ independent of each other is selected from H, CH₂, CH₃, OCH₃, CH₂OH, CH₂OCH₃, OCH₂CH₃, OCH₂CH₂OH, COOH, COOCH₃, COOCH₂CH₃, C(O)NH₂, C(O)N(CH₃)₂, C(O)N(CH₂CH₃)CH₃, or C(O)N(CH₂CH₃)₂;
D₁ to D₃ independent of each other is selected from H, OH, CH₃, CH₂CH₃, CH₂OH, CH₂OCH₃, OCH₂CH₃, OCH₂CH₂OH or OCH₂C₆H₅; and
J₁ to J₃ independent of each other is selected from COOH, P(O)(OH)₂, P(O)(OH)CH₃, P(O)(OH)CH₂CH₃, P(O)(OH)(CH₂)₃CH₃, P(O)(OH)Ph, P(O)(OH)CH₂Ph, P(O)(OH)OCH₂CH₃, CH(OH)CH₃, CH(OH)CH₂OH, C(O)NH₂, C(O)NHCH₃, C(O)NH(CH₂)₂CH₃, OH or H.

Preferred chelators X are residues of diethylenetriaminopentaacetic acid (DTPA), N-[2-[bis(carboxymethyl)amino]-3-(4-ethoxyphenyl)propyl]-N-[2-[bis(carboxymethyl)-amino]ethyl]-L-glycine (EOB-DTPA), N,N-bis[2-[bis(carboxymethyl)amino]-ethyl]-L-glutamic acid (DTPA-Glu), N,N-bis[2-[bis(carboxymethyl)amino]-ethyl]-L-lysine (DTPA-Lys), mono- or bis-amide derivatives of DTPA such as N,N-bis[2-[carboxymethyl[(methylcarbamoyl)methyl]amino]-ethyl]glycine (DTPA-BMA), 4-carboxy-5,8,11-tris(carboxymethyl)-1-phenyl-2oxa-5,8,11-triazamidecan-13-oic acid (BOPTA), DTPA BOPTA, 1,4,7,10-tetraazacyclododecan-1,4,7-triactetic acid (DO3A), 1,4,7,10-tetraazacyclododecan-1,4,7,10-tetraactetic acid (DOTA), ethylenediaminotetraacetic acid (EDTA), 10-(2-hydroxypropyl)-1,4,7,10-tetraazacyclododecan-1,4,7-triacetic acid (HPDO3A), 2-methyl-1,4,7,10-tetraazacyclododecan-1,4,7,10-tetraacetic acid (MCTA), tetramethyl-1,4,7,10-tetraazacyclododecan-1,4,7,10-tetraacetic acid (DOTMA), 3,6,9,15-tetraazabicyclo[9.3.1]pentadeca-1(15), 11,13-triene-3,6,9-triacetic acid (PCTA), PCTA12, cyclo-PCTA12, N,N′Bis(2-aminoethyl)-1,2-ethanediamine (TETA), 1,4,7,10-tetraazacyclotridecane-N,N′,N″,N′″-tetraacetic acid (TRITA), 1,12-dicarbonyl, 15-(4-isothiocyanatobenzyl) 1,4,7,10,13-pentaazacyclohexadecane-N,N′,N″ triaceticacid (HETA), 1,4,7,10-tetraazacyclododecane-N,N′,N″,N′″-tetraacetic acid mono-(N-hydroxysuccinimidyl)ester (DOTA-NHS), N,N′-Bis(2-aminoethyl)-1,2-ethanediamine-N-hydroxy-succinimide ester (TETA-NHS), [(2S,5S,8S,11S)-4,7,10-tris-carboxymethyl-2,5,8,11-tetramethyl-1,4,7,10-tetraazacyclododecan-1-yl]acetic acid (M4DOTA), [(2S,5S,8S,11S)-4,7-bis-carboxymethyl-2,5,8,11-tetramethyl-1,4,7,10-tetraazacyclo-dodecan-1-yl]acetic acid, (M4DO3A), (R)-2-[(2S,5S,8S,11S)-4,7,10-tris-((R)-1-carboxyethyl)-2,5,8,11-tetramethyl-1,4,7,10-tetraazacyclododecan-1-yl]propionic acid (M4DOTMA), 10-Phosphonomethyl-1,4,7,1-O-tetraazacyclododecane-1,4,7 triacetic acid (MPDO3A), hydroxybenzyl-ethylenediamine-diacetic acid (HBED) and N,N′-ethylenebis-[2-(o-hydroxyphenolic)glycine] (EHPG).

The term “residues of . . . ” in the previous paragraph is chosen since the chelator is attached to the remainder of the molecule represented by linear polymers of the present invention. Thus, X is to be seen as a residue. The attachment point of X to said remainder of the molecule represented by linear polymers of the present invention may be any suitable point, e.g. a functional group like a COOH group in a chelator like DTPA, EDTA or DOTA or an amino group in a chelators like DTPA-Lys, but also a non-functional group like a methylene group in chelators like DOTA.

Suitable chelators X and their synthesis are described in e.g. EP-A-071564, EP-A-448191, WO-A-02/48119, U.S. Pat. No. 6,399,043, WO-A-01/51095, EP-A-203962, EP-A-292689, EP-A-425571, EP-A-230893, EP-A-405704, EP-A-290047, U.S. Pat. No. 6,123,920, US-A-2002/0090342, U.S. Pat. No. 6,403,055, WO-A-02/40060, U.S. Pat. No. 6,458,337, U.S. Pat. No. 6,264,914, U.S. Pat. No. 6,221,334, WO-A-95/31444, U.S. Pat. No. 5,573,752, U.S. Pat. No. 5,358,704 and US-A-2002/0127181, the content of which are incorporated herein by reference.

In a more preferred embodiment of the present invention X is a residue selected from DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

In a particularly preferred embodiment X is a residue selected from DTPA, DOTA, BOPTA, DO3A, HPDO3A, DOTMA, PCTA, DTPA BMA, M4DOTA or M4DO3A.

As stated above, in a preferred embodiment of X, i.e. X′, the chelator X forms a complex, i.e. paramagnetic chelate, with a paramagnetic metal ion M. Suitably, M is selected from ions of transition and lanthanide metals, i.e. metals of atomic numbers 21 to 29, 42, 43, 44 or 57 to 71. More preferred, M is a paramagnetic ion of Mn, Fe, Co, Ni, Eu, Gd, Dy, Tm and Yb, particularly preferred a paramagnetic ion of Mn, Fe, Eu, Gd and Dy. Most preferably M is selected from Gd³⁺, Mn²⁺, Fe³⁺, Dy³⁺ and Eu³⁺ with Gd³⁺ being the most preferred paramagnetic ion M.

The linear polymers of the present invention can be linked to vectors to enable targeted MR imaging. By the term “vector” is meant any compound having binding affinity for a specific target, e.g. receptor, tissue or cell type. Linking the linear polymers to vectors can be done by coupling pairs of reactive groups, e.g. aminoxy-aldehyde, azide-triple bond, thiol-alphahaloacetyl, N-alkyl aminoxy-bromocompounds.

One linear polymer of the present invention can be linked to one vector via two reactive groups. Optionally several linear polymers can be linked to one vector via several reactive groups attached to the vector, preferably 2-15 linear polymers can be linked to one single vector. As an example, reaction scheme (1) illustrates three polymers linked to one single vector via three reactive groups on the vector.

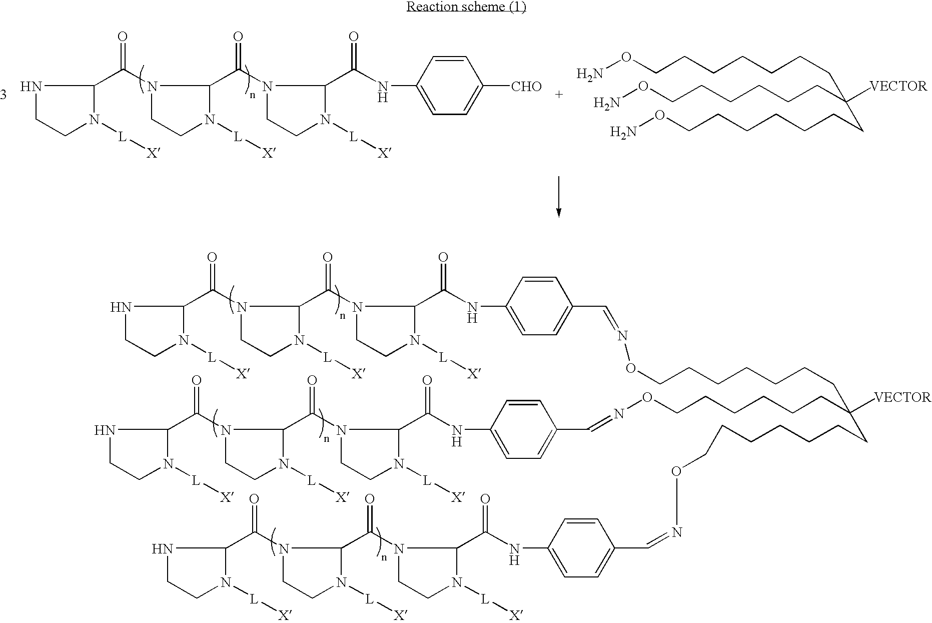

The linear polymers of the present invention can be synthesized by several synthetic pathways known to the skilled artisan.

The linear polymers of the present invention, preferably polymers comprising more than 25 units of formula (I) or (II), can be synthesized by polymerization of said units by head to tail linkages of the amino acid residues A, known in the art e.g. from peptide chemistry, resulting in an amide bond between each of the units.

The monomeric N-carboxyanhydride derivatives can be synthesized and then polymerized according to the following general description with reference to a specific example in reaction scheme (2).

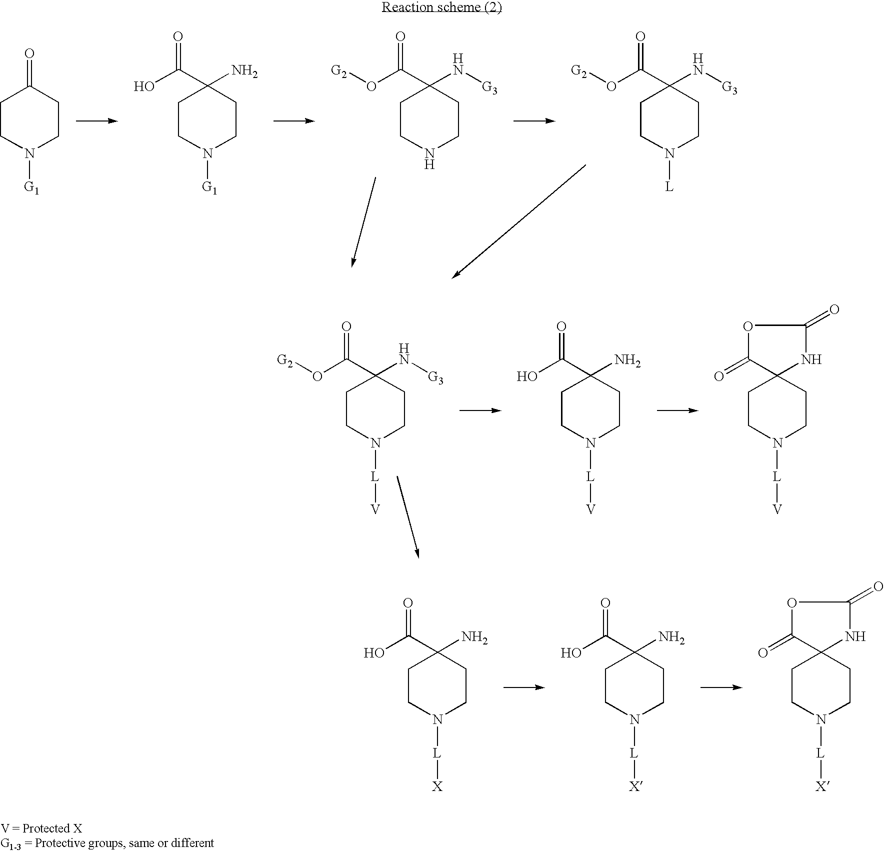

A commercially available cyclic compound containing an aldehyde and a secondary amino group equipped with a protective group (G₁) can be converted into an N-carboxyanhydride derivative containing a metal binding chelate. The aldehyde group can be transformed into an amine and carboxylic acid functionality by reaction with ammonia and hydrogen cyanide followed by acid mediated hydrolysis (Strecker synthesis: A. Strecker. Ann. Chem. Pharm. 75 (1850), p. 27). The formed amine and carboxylate functionality (from now on described as α-amino acid, the corresponding amino acid of residue A) can then be protected using suitable protective groups (G₂) and (G₃), which are chemically inert to the reaction conditions necessary for the deprotection of (G₁). The protective groups (G₂) and (G₃) can for example be the t-butyl group. The protective group (G₁) can then be chemoselectively deprotected to form a free secondary amine, and the obtained compound can then be coupled to a precursor of L-V, where V is a protected form of X, that eventually will form the L-X′ structure. A precursor of L-V will typically include a reactive group or a functional group which can react with amino functionalities, e.g. an acid chloride or an activated ester. Alternatively the formation of the L-V structure can be done stepwise where a precursor to L is reacted with the secondary amine and then the V group is attached to the precursor of L. The precursor of L has a terminal reactive group such as an acid chloride and in addition a leaving group, e.g. chloride. V is then coupled to the L moiety through a replacement reaction with the leaving group.

The protective group (G₁) of the secondary nitrogen is to be chemically inert to the conditions forming the α-amino acid and then to be chemoselectively deprotected in order for the secondary amine functionality to be coupled to the L-V group. An example of a G₁ protective group is a benzyl group. By using suitable reaction conditions (G₂) and (G₃) can be deprotected when transforming V into X. The reaction of X with a suitable metal ion (M) to give X′ is regioselective and the α-amino acid functional groups are left unaffected. The α-amino acid groups can then be transformed into an N-carboxanhydride derivate using a phosgene derivative (phosgene, diphosgene or triphosgene). Alternatively one could form the N-carboxyanhydride derivative from the V substituted compound obtained from chemoselective deprotection of the (G₂) and (G₃) protective groups. The V group will then not be transformed into X′ and X until after polymerization.

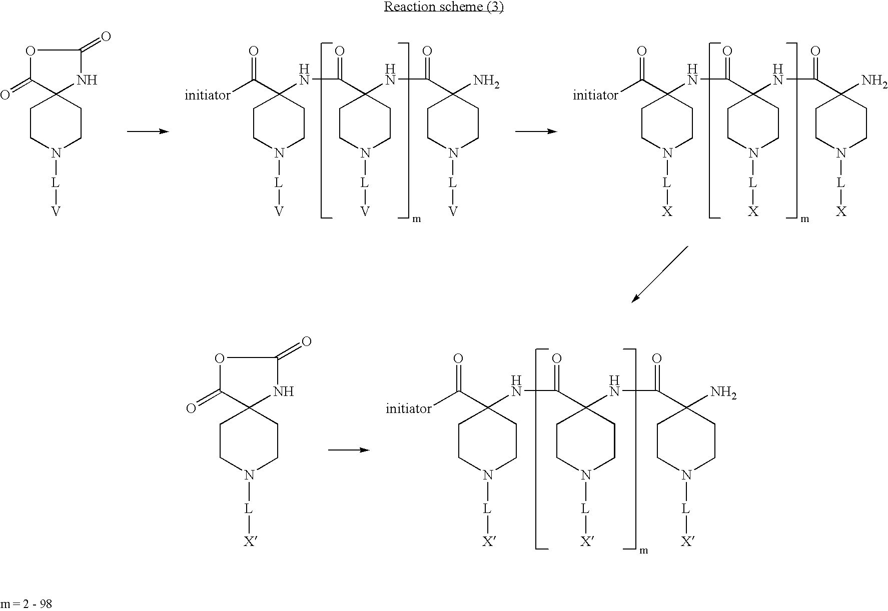

The N-carboxanhydride derivative can be polymerized by addition of a suitable initiator, as illustrated in reaction scheme (3). The initiator is nucleophilic by nature and preferred compounds are various primary amines. The amines can be bifunctional and hence contain a latent reactive group that is stable during the polymerization reaction. This latent reactive group can be activated or chemoselectively reacted after the polymerization in order to couple the polymer to a suitable vector. Examples of a latent reactive group are azides and acetylenes. The molecular weight of the formed polymers can be controlled by adjusting various parameters such as temperature, concentration of monomeric derivative, concentration and nucleophilicity of initiator (T. J. DEMING; J. POLYM. SCI. PART A: POLYM. CHEM.: VOL. 38, 2000). As explained above in case of polymerization of the V containing N-carboxanhydride, the transformation of V into X and finally X′ has to be performed on the polymerized compound.

The linear polymers of the present invention, especially polymers comprising about 4-25 units of formula (I) or (II) respectively, can also be synthesized by solid-phase synthesis. Preferably, said polymers are synthesized using the solid-phase methodology of Merrifield employing an automated peptide synthesizer (J. Am. Chem. Soc., 85: 2149 (1964)).

Synthesis of the linear polymers is based on polymerization of the units (I) or (II) resulting in an amide bond between each conformationally constrained amino acid residue A. The polymerization is done by the sequential addition of units (I) or (II) by linking the protected amino acid residue A to a solid phase support. In one commonly employed method, the α-amino group is suitably protected with acid labile or base labile protecting groups. Following addition and coupling of the first unit (I) or (II), the solid support with the attached unit (I) or (II) is filtered from the unreacted reagents and the α-amino protecting group is removed. The chain is then extended by the addition of a further unit (I) or (II) by linking another protected amino acid residue A to the unprotected amino acid residue attached to the solid support. The solid support with the two units attached is filtered and the α-amino protecting group is removed. This procedure is repeated until the linear polymer comprises the desired number of units (I) or (II).

Generally, to obtain units of formula (II) or linear polymers comprising units of formula (II), X can be transformed into X′ by complex formation with a suitable paramagnetic metal ion M, preferably in the form of its salt (e.g. like Gd(III)acetate or Gd(III)Cl₃). This can be done either before or after the polymerization or synthesis of the polymer.

Thus, another aspect of the invention is a process for the preparation of compounds of formula (II) and preferred embodiments thereof by

(i) reacting residue A with groups L-X or X, wherein A, L and X are as defined as above;
(ii) reacting the reaction product of step (i) with a paramagnetic metal ion, preferably in the form of its salt; and
(iii) polymerizing the reaction product of step (ii) resulting in an amide bond between said As.

### EXAMPLES

The invention is further described in the following examples, which are in no way intended to limit the scope of the invention.

### Preferred Examples of Compounds of Formula (II) are:

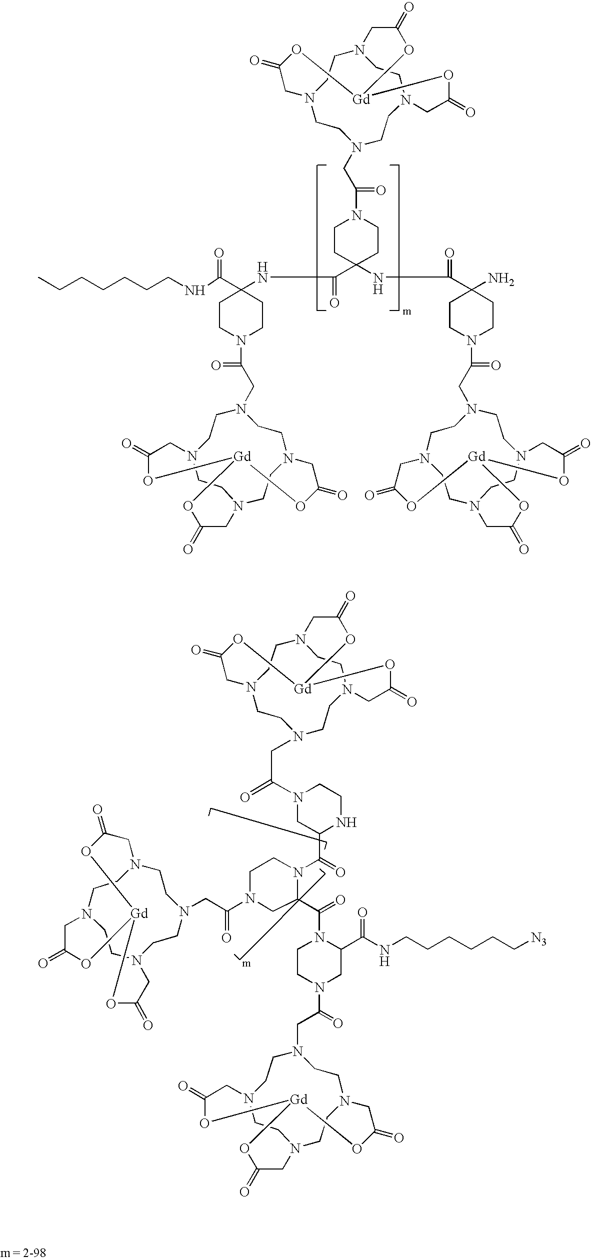

The linear polymers of the present invention comprising units of formula (II) are preferably used as MR contrast agents, e.g. as MR high relaxivity agents, MR imaging agents for tumour imaging or MR blood pool agents. For this purpose, the polymers are formulated with conventional physiologically tolerable carriers like aqueous carriers, e.g. water and buffer solution and optionally excipients.

Hence in a further aspect the present invention provides a composition comprising linear polymers comprising units of formula (II) and at least one physiologically tolerable carrier.

In a further aspect the invention provides a composition comprising linear polymers of the present invention comprising units of formula (II) and at least one physiological tolerable carrier for use as MR imaging contrast agent or MR spectroscopy contrast agent.

To be used as contrast agents for MR imaging or spectroscopy of the human or non-human animal body, said compositions need to be suitable for administration to said body. Suitably, the linear polymers of the present invention comprising units of formula (II) and optionally pharmaceutically acceptable excipients and additives may be suspended or dissolved in at least one physiologically tolerable carrier, e.g. water or buffer solutions. Suitable additives include for example physiologically compatible buffers like tromethamine hydrochloride, chelators such as DTPA, DTPA-BMA, weak complexes of physiologically tolerable ions such as calcium chelates, e.g. calcium DTPA, CaNaDTPA-BMA, compounds of formula (I) or preferred embodiments thereof wherein X forms a complex with Ca²⁺ or CaNa salts of compounds of formula (I) or preferred embodiments thereof, calcium or sodium salts like calcium chloride, calcium ascorbate, calcium gluconate or calcium lactate. Excipients and additives are further described in e.g. WO-A-90/03804, EP-A-463644, EP-A-258616 and U.S. Pat. No. 5,876,695, the content of which are incorporated herein by reference.

Another aspect of the invention is the use of the composition comprising a linear polymer of the present invention comprising units of formula (II) and at least one physiologically tolerable carrier as MR imaging contrast agent or MR spectroscopy contrast agent.

Yet another aspect of the invention is a method of MR imaging and/or MR spectroscopy wherein a composition comprising a linear polymer of the present invention comprising units of formula (II) and at least one physiologically tolerable carrier is administered to a subject and the subject is subjected to an MR procedure wherein MR signals are detected from the subject or parts of the subject into which the composition distributes and optionally MR images and/or MR spectra are generated from said detected signals.

In a preferred embodiment, the subject is a living human or non-human animal body.

In a further preferred embodiment, the composition is administered in an amount which is contrast-enhancing effective, i.e. an amount which is suitable to enhance the contrast in the MR procedure.

In a preferred embodiment, the subject is a living human or non-human animal being and the method of MR imaging and/or MR spectroscopy is a method of MR angiography, more preferred a method of MR peripheral angiography, renal angiography, supra aortic angiography, intercranial angiography or pulmonary angiography.

In another preferred embodiment, the subject is a living human nor non-human animal being and the method of MR imaging and/or MR spectroscopy is a method of MR tumour detection or a method of tumour delineation imaging.

In another aspect, the invention provides a method of MR imaging and/or MR spectroscopy wherein a subject which had been previously administered with a composition comprising a linear polymer of the present invention comprising units of formula (II) and at least one physiologically tolerable carrier is subjected to an MR procedure wherein MR signals are detected from the subject or parts of the subject into which the composition distributes and optionally MR images and/or MR spectra are generated from the detected signals.

The term “previously been administered” means that any step requiring a medically-qualified person to administer the composition to the patient has already been carried out before the method of MR imaging and/or MR spectroscopy according to the invention is commenced.

### SPECIFIC EMBODIMENTS

### Citation of References

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. Linear polymer comprising from 4 to 100 units of the formula (II)
A-L-X′ (II)
wherein
A is the same or different and is a conformationally constrained amino acid residue;
L is absent or present and is the same or different and denotes a linker moiety;
X′ is the same or different and denotes a paramagnetic chelate consisting of a chelator X and a paramagnetic metal ion M; and
wherein said units are linked to each other by amide bonds between said As.

2. Linear polymer according to claim 1 wherein the polymer comprises 5 to 70 of said units.

3. Linear polymer according to claim 1 wherein A is an α,α-substituted amino acid.

4. Linear polymer according to claim 1 wherein A is of the general formula (III) 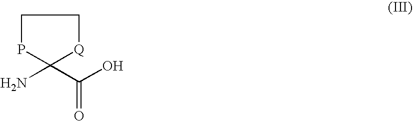
wherein
P is absent or is —CH₂— or —(CH₂)ₙ—NH—; where n is 0 to 6
Q is absent or is —CH₂— or —(CH₂)ₙ—NH—; where n is 0 to 6
where at least one of P or Q is present and is —(CH₂)ₙ—NH—

5. Linear polymer according to claim 1 wherein A is one of

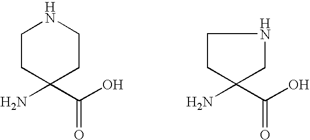

6. Linear polymer according to claim 1 wherein A is of the general formula (IV) 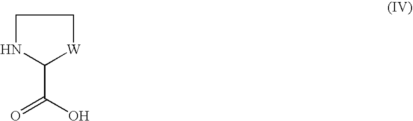
wherein
W is —(CH₂)ₙ—NH—; where n is 0 to 6

7. Linear polymer according to claim 1 wherein A is on of

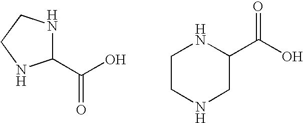

8. Linear polymer according to claim 1 wherein X is a residue selected from DOTA, DTPA, BOPTA, DO3A, HPDO3A, MCTA, DOTMA, DTPA BMA, M4DOTA, M4DO3A, PCTA, TETA, TRITA, HETA, DPDP, EDTA or EDTP.

9. Linear polymer according to claim 1 wherein M is selected from ions of transition and lanthanide metals.

10. Composition comprising the linear polymer of claim 1 and at least one physiological tolerable carrier.

11. Composition according to claim 10 for use as MR imaging contrast agent or MR spectroscopy contrast agent.

12. Method of producing a compound of claim 1 by
(i) reacting residue A with groups L-X or X, wherein A, L and X are as defined in claim 1;
(ii) reacting the reaction product of step (i) with a paramagnetic metal ion, preferably in the form of its salt; and
(iii) polymerizing the reaction product of step (ii) resulting in an amide bond between said As.

13. Use of the composition of claim 10 as MR imaging contrast agent or MR spectroscopy contrast agent.

14. Method of MR imaging and/or MR spectroscopy wherein the composition of claim 10 is administered to a subject and the subject is subjected to an MR procedure wherein MR signals are detected from the subject or parts of the subject into which the composition distributes and optionally MR images and/or MR spectra are generated from said detected signals.

15. Linear polymer comprising from 4 to 100 units of the formula (I)
A-L-X (I)
wherein
A is the same or different and is a conformationally constrained amino acid residue;
L is absent or present and is the same or different and denotes a linker moiety;
X is the same or different and denotes a chelator; and
wherein said units are linked to each other by amide bonds between said As.
